(19) United States
(12) Patent Application Publication
(10) Pub. No.: US 2026/0085340 A1
(43) Pub. Date: Mar. 26, 2026

(54) METHOD FOR SINGLE CELL ANTIMICROBIAL SUSCEPTIBILITY TESTING IN A SUB-DOUBLING TIME

(71) Applicant: The Penn State Research Foundation, University Park, PA (US)

(72) Inventors: Pak Kin WONG, University Park, PA (US); Manuel ROSHARDT, University Park, PA (US)

(21) Appl. No.: 19/114,906

(22) PCT Filed: Sep. 26, 2023
(86) PCT No.: PCT/US2023/075175

§ 371 Date: Mar. 25, 2025

Related U.S. Application Data
(60) Provisional application No. 63/410,089, filed on Sep. 26, 2022.

(51) Int. Cl. C12Q 1/18; G06T 7/00; G06T 7/62; G06T 7/64; G06V 10/44; G06V 10/764; G06V 20/69

(52) U.S. Cl. CPC C12Q 1/18; G06T 7/0012; G06T 7/62; G06T 7/64; G06V 10/44; G06V 10/764; G06V 20/698; G06T 2207/10016; G06T 2207/10056; G06T 2207/20036; G06T 2207/30024; G06V 2201/03

(57) ABSTRACT

Methods and systems for antibacterial susceptibility testing of a bacterium are provided. The method includes exposing a bacterium to an antimicrobial agent. A series of images of the bacterium is captured over time after exposure The series of images are captured during an imaging period. For each image of the series of images, the method includes extracting a value of each feature in a set of morphological features of the bacterium. The set of morphological features includes one or more of area, aspect ratio, length, circularity, perimeter, angularity, curvature, ferret, pole, roundness, sinuosity, width, trajectory, morphology, orientation, solidity, and z-score. A rate of change is calculated for each feature of the set of morphological features during the imaging period. An inhibition status of the bacterium is determined using a machine-learning classifier applied to input data.

### CROSS-REFERENCE TO RELATED APPLICATIONS

This application claims priority to U.S. Provisional Application No. 63/410,089, filed on Sep. 26, 2022, now pending, the disclosure of which is incorporated herein by reference.

### STATEMENT REGARDING FEDERALLY SPONSORED RESEARCH

This invention was made with government support under contract no. R01AI153133 awarded by the National Institutes of Health. The government has certain rights in the invention.

### FIELD OF THE DISCLOSURE

The present disclosure relates to susceptibility testing of microbes, and in particular, susceptibility testing of microbes using imaging.

### BACKGROUND OF THE DISCLOSURE

The emergence of multidrug-resistant pathogens has been a worldwide calamity. Multidrug-resistant bacteria, such as the carbapenemase-producing Enterobacteriaceae, pose an increasing threat to public health due to their high mortality rate and rapid acquisition of resistance to available antimicrobials. Enterobacteriaceae are common causes of nosocomial infection (3%-8% of all nosocomial bacterial infections) and could lead to various life-threatening infections, including severe lung infection, urinary tract infection, and bloodstream infection. Monotherapy with carbapenems like meropenem, an intravenous beta-lactam antibiotic, is often adopted for nosocomial infections of Enterobacteriaceae until antimicrobial susceptibility test (AST) results are available to guide more targeted therapy. However, the prevalence of carbapenemase-producing Enterobacteriaceae is on the rise worldwide, for example, 12.4% in Libya and 13.4% in 2017 to 14.6% in 2019 in China. In the absence of a rapid method to determine antimicrobial susceptibility, empirical use of antibiotics is warranted. Inappropriate antibiotic treatment results in almost twice higher mortality rate in infected patients and accelerates the emergence and spread of superbugs. Therefore, rapid determination of antimicrobial susceptibility to guide treatment is crucial to save lives and curb the widespread of multidrug-resistant pathogens. There continues to be a need for improved approaches for determining antimicrobial susceptibility.

### BRIEF SUMMARY OF THE DISCLOSURE

Multidrug-resistant bacteria are a global public health threat. Rapid determination of a bacterium's resistance to antimicrobials is a major clinical unmet need in the diagnosis of bacterial infections. The present disclosure provides a method for morphometric antimicrobial susceptibility testing (referred to herein as “MorphoAST”). The described approach provides an image-based machine learning workflow that is used for rapid determination of antimicrobial susceptibility by single cell morphological analysis in a sub-doubling time. By capturing dynamic single cell morphological features of over twenty-eight thousand cells, we evaluated strategies based on time and concentration differentials for classifying the susceptibility of Klebsiella pneumoniae to meropenem and predicting their minimum inhibitory concentrations (MIC). The classifiers achieved as high as 97% accuracy in 20 minutes (two-fifths of the doubling time) and reached over 99% accuracy within 50 minutes (one double time) in predicting the antimicrobial response. A regression model based on the concentration differential of individual cells predicted the MIC with >97% categorical agreement and 100% essential agreement. When tested against cells from an unseen strain, the regressor achieved a categorical agreement of 91.9% with a very major error of 0.1%. The ability to predict antimicrobial responsiveness in a fraction of the doubling is expected to have significant implications in the management of bacterial infections.

In contrast to the presently described approaches, phenotypic AST, such as broth microdilution and Kirby-Bauer disk diffusion test, evaluates the ability of an antimicrobial to inhibit bacteria growth and has the gold standard for determining antimicrobial susceptibility. The turbidity of liquid media or the formation of bacterial colonies provides a measure of bacteria growth with the presence of an antimicrobial and generates quantitative MIC. Nevertheless, phenotypic AST, which relies on bacterial replication for 18 hours or more, is unable to accommodate a rapid turnaround. Further, single cell imaging analysis is an emerging strategy that offers the possibility of reducing the turnaround time and improving the diagnostic resolution. By visualizing the replication of individual cells, the response of bacteria to antimicrobials can be in principle reduced to one or a few doubling times of the bacteria. However, rapid AST techniques that rely on the area occupied by the cells as a measure of the growth could mistake a transient increase in cell sizes due to antimicrobial tolerance as growth. Phenotypic variants, drug accumulation, and growth phase can also introduce uncertainties in rapid AST assays. Because of these reasons, existing imaging-based approaches often require at least 2 hours, if not 4 hours or more, to deliver reliable results, especially for slow-growing and fastidious bacteria. While there are numerous previous examples of rapid AST, the disclosure addresses an unmet need by providing an assay that can deliver AST results (1) rapidly in a point-of-care timeframe, (2) quantitatively with MIC determination, (3) efficiently with a small inoculum size, and (4) is suitable for direct use with primary clinical samples.

In an aspect, the present disclosure provides a method for antibacterial susceptibility testing of a bacterium. The method includes exposing a bacterium to an antimicrobial agent. A series of images of the bacterium is captured over time after exposure (i.e., exposure to the antimicrobial agent). The series of images are captured during an imaging period. The imaging period may be less than the doubling time for the bacterium. For each image of the series of images, the method includes extracting a value of each feature in a set of morphological features of the bacterium. The set of morphological features includes one or more of area, aspect ratio, length, circularity, perimeter, angularity, curvature, ferret, pole, roundness, sinuosity, width, trajectory, morphology, orientation, solidity, and z-score. For example, the set of morphological features may be made up of area, aspect ratio, length, circularity, and perimeter.

A rate of change is calculated for each feature of the set of morphological features during the imaging period. In some embodiments, the rate of change is calculated by fitting a curve of the values for a feature with an exponential function and calculating a coefficient (i.e., the feature changing rate coefficient. For example, the exponential function may be ƒ(t)=A1+A2e^{t/R}, where ƒ(t) is the feature as a function of time, A1 and A2 are constants, and R is the feature changing rate coefficient.

An inhibition status of the bacterium is determined using a machine-learning classifier applied to input data. The input data includes the rate of change for each feature of the set of morphological features. In some embodiments, the input data may further include a concentration of the antimicrobial agent. The machine learning classifier may be, for example, a k-nearest neighbor classifier, a multilayer perceptron classifier, a random forest regressor, or other classifier, or combinations.

In some embodiments, the method includes repeating the steps for various concentrations of antimicrobial agents.

In another aspect, the present disclosure provides a system for antibacterial susceptibility testing of a single-cell sample. The system includes a sample holder and an image sensor positioned to obtain one or more images of a sample held in the sample holder. A processor is in electronic communication with the image sensor. The processor is configured to perform any of the methods disclosed herein. For example, the processor may be configured to: receive from the image sensor a series of images of a sample over time after exposure to an antimicrobial agent; extract, for each image of the series of images, values of each feature in a set of morphological features of the sample, wherein the set of morphological features comprises one or more of area, aspect ratio, length, circularity, perimeter, angularity, curvature, ferret, pole, roundness, sinuosity, width, trajectory, and z-score; calculate a rate of change for each feature of the set of morphological features during the imaging period; and determine an inhibition status of the sample using a machine-learning classifier applied to input data comprising the rate of change for each feature of the set of morphological features.

### DESCRIPTION OF THE DRAWINGS

For a fuller understanding of the nature and objects of the disclosure, reference should be made to the following detailed description taken in conjunction with the accompanying drawings, in which:

FIG. 1. A schematic flowchart of the single cell MorphoAST workflow for susceptibility classification and minimum inhibitory concentration (MIC) prediction. (A) The workflow starts with time-lapse imaging of individual bacteria under various antimicrobial concentrations. The morphological features of the bacteria are extracted automatically using MicrobeJ, an ImageJ plugin. (B) In the time differential approach, the feature changing rates are extracted by exponential curve fitting from the time-lapse data. (D-E) The feature changing rate datasets are applied to train an artificial neural network to create a classification model.

FIG. 2. Representative results of single cell MorphoAST of K. pneumoniae treated with meropenem, an intravenous β-lactam antimicrobial. (A) Time-lapse images of K. pneumoniae (KP0142) treated with (top) 0 and (bottom) 50 μg/ml meropenem. Time-lapse imaging was performed 15 minutes after mixing with meropenem at a 5-minute interval. The MIC of the strain was 2 μg/ml, and cell division was completely inhibited with 50 μg/ml. Bulging the cells was observed only in the meropenem case. (B) Zoom-in views of KP0142 treated with 50 μg/ml meropenem. (C) Morphological analysis with MicrobeJ for extracting cell features, such as area, length, width, circularity, curvature, sinuosity, angularity, and solidity. (D) Examples of two bacterial growth curves for extracting the area changing rate. (E) Distributions of area and length changing rates of individual K. pneumoniae exposed to various meropenem concentrations.

FIG. 3. Classification accuracy of bacteria groups using the time differential approach. (A) Principal component analysis plots of dynamic features of 1338 bacteria from various bacteria strains and antimicrobial concentrations. The data are labeled as 1 (light gray-division or resistant) or 0 (dark gray-no division or susceptible). Bacteria are randomly selected from the same strain-concentration combination to form a group, and the dynamic features are averaged for groups of 1, 3, 5, and 7 bacteria. (B-C) Prediction accuracy obtained by (B) k-nearest neighbors and (C) artificial neural network algorithms for different group sizes. For single bacteria, the accuracy was slightly higher for the artificial neural network (˜90%). With groups of seven bacteria, both models obtained over 99% accuracy. The accuracy values are average of 10 repetitions. (D) Evolution of confusion matrices of neural network classifiers with groups of 1 to 7 bacteria. Both the major error (true susceptible and predicted resistant) and the very major error (true resistant and predicted susceptible) were reduced with increasing number of bacteria.

FIG. 4. Classification accuracy of sub-doubling time susceptibility prediction using the time differential approach. (A) Principal component analysis plots of feature changing rates with 20-50 minutes of antimicrobial exposure, which correspond 5 to 35 minutes duration of dynamic data. The data are labeled as 1 (light gray-division or resistant) or 0 (dark gray-no division or susceptible) at the strain-concentration combination with groups of seven bacteria. (B-C) Prediction accuracy obtained by (B) k-nearest neighbors and (C) artificial neural network algorithms for different durations of data. The accuracy was approximately 80% for a 5-min duration, and the value improved to over 99% accuracy with a 35-minute duration of data. The accuracy values are average of 10 repetitions. (D) Evolution of confusion matrices of neural network classifiers with groups of 20-50 minutes of antimicrobial exposure. Both the major error (true susceptible and predicted resistant) and the very major error (true resistant and predicted susceptible) were reduced with the duration of data.

FIG. 5. A chart depicting a method according to an embodiment of the present disclosure.

FIG. 6. compares three strains (KP0016-Susceptible, KP0143-Resistant, and KP0142-Intermediate) at the early time points.

FIG. 7. Graphs showing extracted feature values using an experimental embodiment.

FIG. 8. An illustration depicting a system according to another embodiment of the present disclosure.

FIG. 9. Minimum inhibitory concentration prediction with a random forest regressor using the concentration differential approach. (A) Evaluation of the regression model with root-mean-square error (RMSE), R-squared and mean absolute error (MAE) values. The regression model trained on the best set of 19 strains was assessed for the lowest RMSE and MAE and highest R-squared values. With historical data and accumulated time, the model performs better as seen in all metrics. (B) Mode of predicted MIC for each unseen test strain in comparison to the experimental MIC for each strain. The regressor achieved 100% essential agreement in the predicted mode MIC within 50 minutes. (C) Histogram of the log 2 dilution of the ratio between experimental MIC (MICexp) and CDC reported MIC (MICp) for all the cells in the test strains. ˜85.1% of all cells had predicted MIC within one-two fold dilution from the experimental MIC after 50 minutes of exposure to meropenem. (D) MIC prediction and susceptibility classification of two clinical isolates from patients with urinary tract infections with 922 (VA_1) and 648 (VA_2) cells imaged across multiple concentrations of meropenem. The model predicts the mode MIC for VA_1 and VA_2 to be 1 μg/mL and accurately classified both to be susceptible. (E) Histogram of the log 2 dilution of the ratio between reported MIC (MICᵣₑₚ) and CDC reported MIC (MIC_{pred}). Extensive heterogeneity in the populations of the clinical isolates, even within 50 minutes, only ˜40% of the cells from the two isolates starts to show susceptibility phenotype.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Ranges of values are disclosed herein. Unless otherwise stated, the ranges include all values to the magnitude of the smallest value (either lower limit value or upper limit value) and ranges between the values of the stated range. These include but are not limited to all values for bacterial detection sensitivity and specificity, all time periods, temperatures, bacteria morphological features, reagents, volumes, sizes and all methods of using the devices and system described herein.

The disclosure includes all compositions of matter, and all method steps described herein. The methods may include all steps consecutively as described, or steps may be omitted, or their order changed. All compositions of matter formed during the methods described herein are included within the scope of this disclosure. Any composition may comprise or consist of any physical matter described herein. Any method may comprise or consist of steps described herein. Any composition of matter or step may be expressly excluded from the scope of any claims presented with this application or patent.

The described report MorphoAST approach provides an image-based machine learning workflow. The described device and methods are suitable for rapid antimicrobial susceptibility testing by single cell morphological analysis in sub-doubling times. The disclosure is demonstrated in non-limiting embodiments by measuring the growth profiles of 1338 bacteria under different antibiotic concentrations. The dynamic morphological features are extracted to train machine learning classifiers that are used to predict the antibiotic resistance of individual bacteria before division. In embodiments, the described approach results in 90% accuracy for predicating the growth response of individual bacteria to antibiotics and achieves over 99% accuracy with groups of seven bacteria. In non-limiting examples, rapid antimicrobial susceptibility testing was demonstrated by analyzing the dynamic morphological features with as short as 5 minutes (approximately one-tenth of the doubling time). The described ability to predict antibiotic responsiveness in a sub-doubling time is expected to provide significant advances in diagnosing slow-growing bacteria and accelerating the clinical diagnosis of bacterial infections.

The described approach can be used with any anti-bacterial agent. In embodiments, the disclosure relates determining AST to any of narrow-spectrum beta-lactam antibiotics of the penicillin class of antibiotics. In embodiments, the antibiotic comprises ciprofloxacin. In embodiments, the antibiotic is methicillin (e.g., meticillin or oxacillin), or flucloxacillin, or dicloxacillin, or some or all of these antibiotics. In embodiments, the antibiotic is vancomycin. In embodiments, the antibiotic is linezolid (ZYVOX), daptomycin (CUBICIN), quinupristin/dalfopristin (SYNERCID). In embodiments, resistance (or susceptibility) to an antimicrobial peptide is used. In embodiments, resistance to any of the following types of antimicrobial agent is determined: Arsphenamine, Penicillin, Sulfonamide, Cephalosporin, Chlortetracycline, Polymyxin, Chlorampheniol, Nitrofurans, Bacitracin, Streptomycin, Metronidazole, Rifamycin, Novobiocin, Cycloserine, Streptogramin, Vancomycin, Isoniazid, Erythromycin, Pleuromutilin, Fosfomycin, Fusidic acid, Lincomycin, Trimethoprim, Nalidixic acid, Oxazolidinone, Carbapenem, Fidaxomicin, Mupirocin, Daptomycin, Monobactam, Bedaquiline, or Delamanid.

The disclosure is suitable for testing any type of bacteria. In embodiments, the bacteria are any of E. coli, P. aeruginosa, K. pneumoniae, M. tuberculosis, any type of Staphylococcus, any type of Enterococcus, or a combination thereof.

In certain embodiments a result obtained from using a method and/or device and/or system of this disclosure can be compared to any suitable reference, examples of which include but are not limited control sample(s), a standardized curve(s), and/or experimentally designed controls such as a known input bacteria value used to normalize experimental data for qualitative or quantitative determination of the presence, absence, amount, or type of bacteria, or a cutoff value. A reference value may also be depicted as an area on a graph. In embodiments the disclosure provides for an internal control that can be used to normalize a result.

In certain embodiments a result based on a determination of the presence, absence, amount, type of bacteria, antibiotic resistance thereof, or a combination thereof, using an approach/device of this disclosure is obtained and is fixed in a tangible medium of expression, such as a digital file, and/or is saved on a portable memory device, or on a hard drive, or is communicated to a web-based or cloud-based storage system. The determination can be communicated to a health care provider for diagnosing or aiding in a diagnosis, such as of a bacterial infection, and/or for recommending or not recommending a particular antibiotic, or for monitoring or modifying a therapeutic or prophylactic approach for any bacterial infection. The disclosure includes determining that bacteria in a sample obtained from an individual are sensitive to one or more antibiotics, and administering an antibiotic to which the bacteria are sensitive to the individual from whom the sample was obtained.

In embodiments, the disclosure provides for monitoring treatment of an individual, such as by testing a first sample for the presence of bacteria, treating the individual with an antimicrobial agent, and testing a second sample using the described approach to determine if the antimicrobial treatment is effective.

In embodiments, efficacy of candidate antimicrobial agents can be used by, for example, exposing a population of bacteria to the candidate antimicrobial agent, and testing the population using any method and/or device described herein to determine if the test agent is capable of inhibiting the growth and/or killing the bacteria.

In certain examples the disclosure comprises an article of manufacture, which in embodiments can also be considered kits. The article of manufacture comprises at least one component for use in the bacterial analysis described herein, and packaging. The packaging can contain any device described herein. In various embodiments, the article of manufacture includes printed material. The printed material can be part of the packaging, or it can be provided on a label, or as paper insert or other written material included with the packaging. The printed material provides information on the contents of the package, and instructs user how to use the package contents for bacteria analysis.

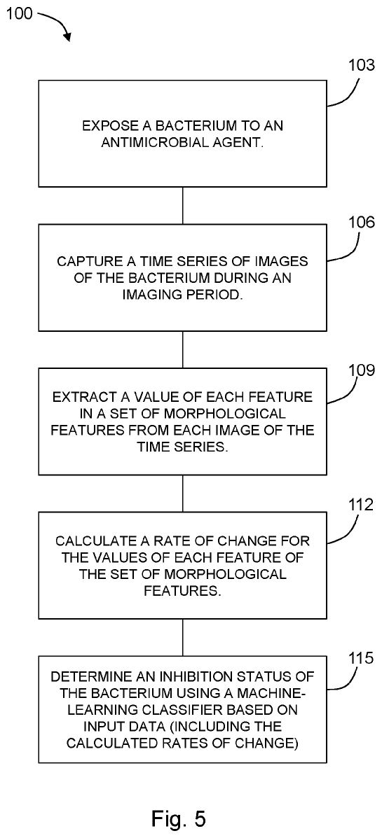

An overview of a workflow of the disclosure is provided in FIG. 5. The workflow depicted is a non-limiting overview of the approach that was used to produce the data presented in other figures that accompany this disclosure.

With reference to FIG. 5, the present disclosure may be embodied as a method 100 for antimicrobial susceptibility testing. For convenience, the present disclosure will reference the non-limiting example of antibacterial susceptibility testing of a bacterium. The method 100 includes exposing 103 a bacterium to an antimicrobial agent. A series of images are captured 106 over time after exposure 103. The series of images are captured 106 during an “imaging period.” The imaging period may be less than the doubling time for the bacterium (for example, less than 100%, 80%, 60%, 50%, 40%, 20%, or 10% of the doubling time).

Values for each feature in a set of morphological features are extracted 109 from each image of the series of images. The morphological features include one or more of area, aspect ratio, length, circularity, perimeter, angularity, curvature, ferret, pole, roundness, sinuosity, width, trajectory, morphology, orientation, solidity, and z-score. In some embodiments, the morphological features include area, aspect ratio, length, circularity, and perimeter. The method 100 includes calculating 112 a rate of change for each feature of the set of morphological features during the imaging period. The rate of change may be calculated 112 by fitting a curve of the values for the respective feature with an exponential function and calculating a coefficient (i.e., a feature changing rate coefficient). For example, the exponential function may be:

$$f ( t ) = A 1 + A 2 {e}^{t / R} \qquad ( 1 )$$

where ƒ(t) is the feature as a function of time, A1 and A2 are constants, R is the feature changing rate (i.e., the coefficient).

A machine-learning classifier is used to determine 115 an inhibition status of the bacterium. The machine-learning classifier is applied to input data which includes the calculated 112 rate of change for each feature of the set of morphological features. The input data may further include a concentration of the antimicrobial agent. Other data may be included in the input data, such as, for example, time of tracking (input period) and bacterial strain. The inhibition status may be determined 115 to be, for example, resistant/susceptible, division/no division, etc. The machine-learning classifier may be, for example, an artificial neural network. The machine-learning classifier may be, for example, a k-nearest neighbor classifier, a multilayer perceptron classifier, a random forest regressor, or other classifier, or combinations of classifiers. The machine learning classifier may be trained to classify an inhibition status based on a training set of morphological feature rates of change.

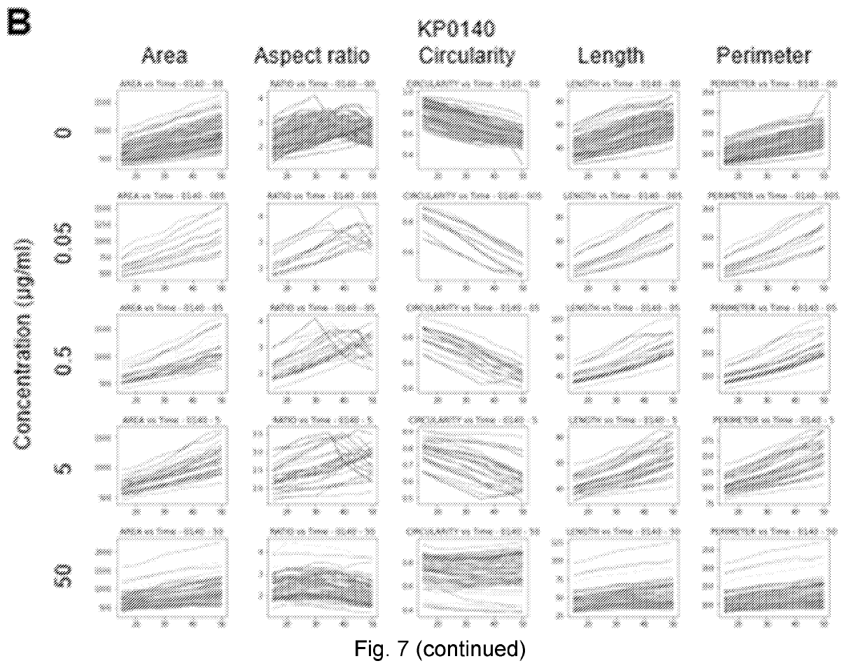

With reference to FIG. 8, in another aspect, the present disclosure may be embodied as a system 10 for antibacterial susceptibility testing of a single-cell sample. The system 10 may include a sample holder 12, an image sensor 14 positioned to obtain one or more images of a sample 90 held in the sample holder 12, and a processor 20 in communication with the image sensor 14. The processor is configured to perform any of the methods disclosed herein. For example, the processor may be configured to receive from the image sensor a series of images of a sample over time after exposure to an antimicrobial agent; extract, for each image of the series of images, values of each feature in a set of morphological features of the sample, wherein the set of morphological features comprises one or more of area, aspect ratio, length, circularity, perimeter, angularity, curvature, ferret, pole, roundness, sinuosity, width, trajectory, and z-score; calculate a rate of change for each feature of the set of morphological features during the imaging period; and determine an inhibition status of the sample (for example, resistant/susceptible, division/no division, etc.) using a machine-learning classifier applied to input data comprising the rate of change for each feature of the set of morphological features.

The processor may be in communication with and/or include a memory. The memory can be, for example, a random-access memory (RAM) (e.g., a dynamic RAM, a static RAM), a flash memory, a removable memory, and/or so forth. In some instances, instructions associated with performing the operations described herein (e.g., operate an image sensor, capture a series of images) can be stored within the memory and/or a storage medium (which, in some embodiments, includes a database in which the instructions are stored) and the instructions are executed at the processor.

In some instances, the processor includes one or more modules and/or components. Each module/component executed by the processor can be any combination of hardware-based module/component (e.g., a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), and/or the like.

In another aspect, the present disclosure may be embodied as a non-transitory computer-readable medium having stored thereon a computer program for instructing a computer to perform any of the methods described herein. For example, the non-transitory medium may have instructions to receive from the image sensor a series of images of a sample over time after exposure to an antimicrobial agent; extract, for each image of the series of images, values of each feature in a set of morphological features of the sample, wherein the set of morphological features comprises one or more of area, aspect ratio, length, circularity, perimeter, angularity, curvature, ferret, pole, roundness, sinuosity, width, trajectory, and z-score; calculate a rate of change for each feature of the set of morphological features during the imaging period; and determine an inhibition status of the sample (for example, resistant/susceptible, division/no division, etc.) using a machine-learning classifier applied to input data comprising the rate of change for each feature of the set of morphological features.

Some instances described herein relate to a computer storage product with a non-transitory computer-readable medium (which can also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other instances described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, instances may be implemented using Java, C++, .NET, or other programming languages (e.g., object-oriented programming languages) and development tools. Additional examples of computer code include, but are not limited to, control signals, encrypted code, and compressed code.

The disclosure provides a strategy for rapidly determining bacteria response to antimicrobials by monitoring their morphological changes. Bacteria undergo a wide variety of morphological changes in response to the environment. These changes, such as filamentation, bulging, and lysis, indicate stress in bacteria and have been applied for investigating the mechanisms of action of antimicrobials. For instance, distinct morphological transformations could be induced in Escherichia coli depending on the type of beta-lactam antimicrobials. Pseudomonas aeruginosa, which are known to be highly tolerant against beta-lactams, undergo a transition from rod-shaped to viable spherical cells when treated with meropenem. The minute change in the bacterial morphology could be indicative of the bacterial response to antimicrobials prior to cell replication. While previous examples of bacterial analysis at the single cell level have emerged, the potential of single-cell morphological analysis for rapid AST and MIC determination in the present disclosure takes into account the dynamic, multiparametric morphological features of bacteria.

### Experimental Embodiment

Herein, we demonstrate a non-limiting embodiment of the present disclosure embodied as a machine learning workflow, termed morphometric antimicrobial susceptibility test (MorphoAST), for single cell AST and MIC quantification in a fraction of the bacterial doubling time. The workflow combines single cell imaging, computer vision feature extraction, and supervised learning models for predicting the response of bacteria to antimicrobials. We measured dynamic morphological features of individual Klebsiella pneumoniae in the presence of meropenem. We processed the data using time and concentration differential strategies and trained machine learning models to predict the antimicrobial response and MIC. The models were validated by cross-validation and left-out data to access their predictive power. A test dataset with cells from an unseen strain was tested to provide unbiased evaluation of the trained model. The results were reported according to the CLSI performance standards for antimicrobial susceptibility testing (M100) guideline.

### Materials and Methods

### Bacteria Culture

Klebsiella pneumoniae isolates (listed in Table 1 below) from the CDC & FDA Antimicrobial Resistance (AR) bank and American Type Culture Collective (ATCC) were grown in Mueller Hinton Broth (MHB) at 37° C. overnight. The next day, cells were sub-cultured in fresh MHB media for three hours (growth phase) to a density equivalent to OD₆₀₀=0.5. Bacteria cells (10 μL) were treated with 10 μL meropenem at varying final concentrations of 0, 0.05, 0.5, 5, and 50 μg/mL to ascertain the physiological responses of cells. A subset of these isolates was treated with finer range of antimicrobial concentrations (0.02, 0.04, 0.016, 0.032, 0.064, 0.125, 0.5, 1, 2 μg/mL) to train the algorithm for quantitative prediction of MIC. The MICs of the isolates were obtained from the CDC and independently confirmed using the broth microdilution method according to the CDC guideline.

TABLE 1
Klebsiella pneumoniae strains from the ATCC and CDC/FDA Antimicrobial
Resistance Isolate Bank (Enterobacterales carbapenemase diversity and
breakpoint panel). Strains 1-10 were obtained from the CDC/FDA Antimicrobial
Resistance Isolate Bank. Strains 11-12 were obtained from ATCC.

| | | | | | |
|---|---|---|---|---|---|
| | KP | Meropenem | | Resistance | Sequence |
| No | Strains | Susc. | MIC | Mechanisms | Accession # |

AR Bank#

| | | | | | |
|---|---|---|---|---|---|
| 1 | 0016 | S | <0.5 | LEN16 | SAMN04014857 |
| 2 | 0140 | I | 2 | aac(3)-IId, aac(6′)Ib-cr, aadA2, | SAMN04014981 |
| | | | | armA, catA1, CTX-M-15, | |
| | | | | dfrA12, dfrA14, OmpK35, OXA- | |
| | | | | 181, SHV-26, sul1, sul2, tet(A) | |
| 3 | 0153 | R | >8 | aac(3)-IId, aadA2, armA, cmlA1, | SAMN04014994 |
| | | | | CTX-M-15, dfrA1, dfrA12, | |
| | | | | dfrA14, fosA, mph(E), | |
| | | | | msr(E), NDM-1, oqxA, OXA-1, | |
| | | | | OXA-232, OXA-9, strA, strB, | |
| | | | | sul1, sul2, TEM-1A | |
| 4 | 0142 | I | 2 | aac(3)-IId, aac(6′)Ib-cr, aadA2, | SAMN04014983 |
| | | | | armA, CTX-M-15, dfrA12, | |
| | | | | dfrA14, OmpK35, OXA-181, | |
| | | | | SHV-26, sul1, sul2, tet(A) | |
| 5 | 0120 | R | >8 | aac(6′)-33, aac(6′)-Ib, aadA2, | SAMN04014961 |
| | | | | aadB, aph(3′)-Ia, dfrA12, KPC-2, | |
| | | | | mph(A), OmpK35, oqxA, oqxA, | |
| | | | | oqxB, sul1, sul2, TEM-1D | |
| 6 | 0010 | S | 1 | CMY-94, EMRD, fosA, KDEA, | SAMN04014851 |
| | | | | Omp35, OmpK35, oqxA, | |
| | | | | oqxB14, SHV-1 | |
| 7 | 0160 | R | >8 | fosA, oqxA, oqxA, oqxB, OXA- | SAMN04015001 |
| | | | | 48, SHV-11 | |
| 8 | 0028 | S | <0.5 | aph(3′)-Ia, OXY-1-1 | SAMN04014869 |
| 9 | 0147 | S | 0.5 | aadB, aph(3′)-Ic, dfrA3b, KPC-3, | SAMN04014988 |
| | | | | OXY-1-4, sul1 | |
| 10 | 0143 | R | >8 | aac(3)-IId, aac(6′)-Ib, armA, | SAMN04014984 |
| | | | | ARR-3, catA1, cmlA1, CMY-4, | |
| | | | | CTX-M-15, dfrA1, fosA, | |
| | | | | mph(E), msr(E), NDM-1, oqxA, | |
| | | | | oqxA, OXA-9, strA, strB, sul2, | |
| | | | | TEM-1A | |
| | ATCC | | | | |
| 11 | BAA-1705 | R | >8 | blaKPC | |
| 12 | 700603 | S | <=0.25 | SHV-18 | |

### Bacteria Imaging

Bacteria cells and meropenem in liquid media are briefly vortexed prior to mounting on 1% (v/v) UltraPure agarose pad (10×20 mm, Invitrogen) on a microslide covered with a glass coverslip (#1.5, 22×30 mm). Bacteria imaging was performed using a Nikon Ti2-E inverted microscope equipped with a DS-Qi2 CMOS camera and an Okolab stage-top temperature control chamber (37° C., 5% CO₂). Images were acquired using a Nikon CFI Plan Apochromat 1 DM 100× oil objective lens and an external Phase Contrast (Ph3) module. Each isolate was observed over a period of 75 minutes (15-90 minutes after mixing with the antimicrobial).

### Feature Extraction

Time-series images were stacked and corrected for shifts in the time series using the Template Matching and Slice Alignment ImageJ plugin. Each individual stack was then analyzed using the MicrobeJ plugin for ImageJ. The plugin, designed for the detection and analysis of bacterial cells, uses computer vision algorithms to automatically identify a cell and determine a suite of morphological features, such as its area, length, circularity, and perimeter (see Table 2 below). The tracking data were summarized as a table and exported as a comma-separated values (.csv) file for further analysis.

TABLE 2
Size shape and size parameters generated by MicrobeJ

| No. | Parameters |
|---|---|
| 1 | angularity |
| 2 | area |
| 3 | aspect ratio |
| 4 | circularity |
| 5 | curvature |
| 6 | feret |
| 7 | feret.max |
| 8 | feret.min |
| 9 | length |
| 10 | morphology |
| 11 | orientation |
| 12 | perimeter |
| 13 | pole |
| 14 | roundness |
| 15 | sinuosity |
| 16 | solidity |
| 17 | width |
| 18 | width.mean |
| 19 | width.stdev |
| 20 | trajectory |
| 21 | zscore |

### Data Analysis and Machine Learning

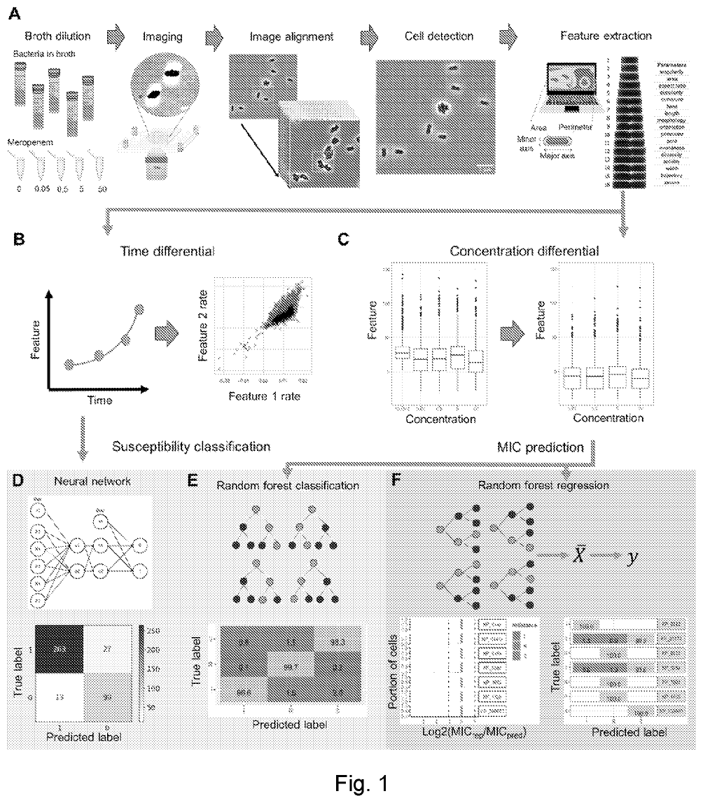

The morphological data were analyzed to predict the susceptibility and MIC of the bacterial strain before the average doubling time. Two differential strategies based on the time-dependent or antibiotic concentration-dependent changes of the bacteria were applied. The processed data were then applied for susceptibility classification and MIC prediction. A schematic of the workflow is shown in FIG. 1.

In the time differential (or dynamic) approach, processed image stacks for Klebsiella pneumoniae isolates (KP016, KP0140, KP1705, KP0153, and KP0142) at various antimicrobial concentrations were evaluated. The cells were labeled as division (resistant) or no division (susceptible). Data analyses were performed using Python with data analysis and machine learning libraries. To explore AST in a sub-doubling time, time-lapse data from the first 35 minutes (corresponding to 15 to 50 minutes after antimicrobial exposure) at a 5-minute interval were analyzed. Erroneous data (e.g., missing bacteria) were detected and removed.

Feature changing rates of individual cells were determined by fitting the time-lapse data with the exponential function. The area, aspect ratio, circularity, length, and perimeter were the most relevant dynamic features. These feature changing rates for each cell, along with the meropenem concentration, time of tracking, the bacterial strain, and the label for growing/not growing, were used to train the k-nearest neighbor and artificial neural network (MPLClassifier) models from the scikit-learn library. For the k-nearest neighbor model, k was optimal at 20 in the experimental embodiment. An advantageous structure of the artificial neural network was found at three hidden layers with five neurons each, even though a simple grid (two hidden layers with two neurons each) was sufficient for well-separated data (e.g., 50-min data). The accuracy of each model was obtained by averaging the values of 10 runs.

### Results

### The MorphoAST Workflow for Rapid AST

We developed a non-limiting example machine learning workflow for rapid AST in a sub-doubling time of the bacteria. The MorphoAST workflow started with imaging of individual bacteria under various antimicrobial concentrations (FIG. 1A). Cells were grown to log phase and treated with varying concentrations of meropenem. Cells without any antimicrobials were imaged in the same manner as controls for the experiment. Live bacteria were mounted on an agarose pad to minimize cell movement due to cell motility and Brownian motion. Time-lapse images were taken every 5 minutes. Images were aligned to match the location of individual cells at each frame and then subject to MicrobeJ analysis for extracting morphological features. A total of 21 parameters (Table 2) describing the cellular dimension and orientation were generated. The data were analyzed either fitting with an exponential function to extract the feature changing rates (FIG. 1B) or normalized against the untreated control to extract the feature differentials (FIG. 1C). The data were applied to train and validate machine learning classifiers to predict the antimicrobial response (FIG. 1D) and regressors to predict the MIC of the bacterial strain against the antimicrobial (FIG. 1E).

### Single Cell Imaging and Feature Extraction

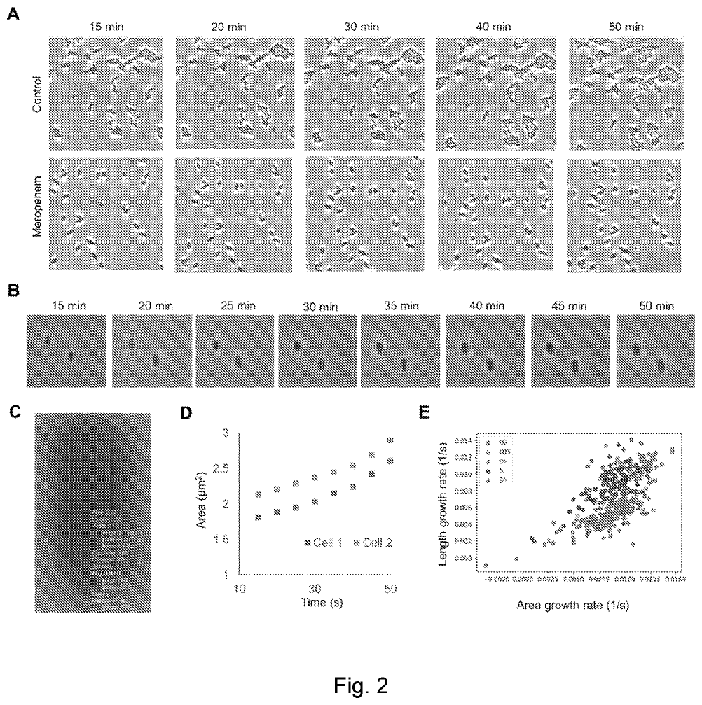

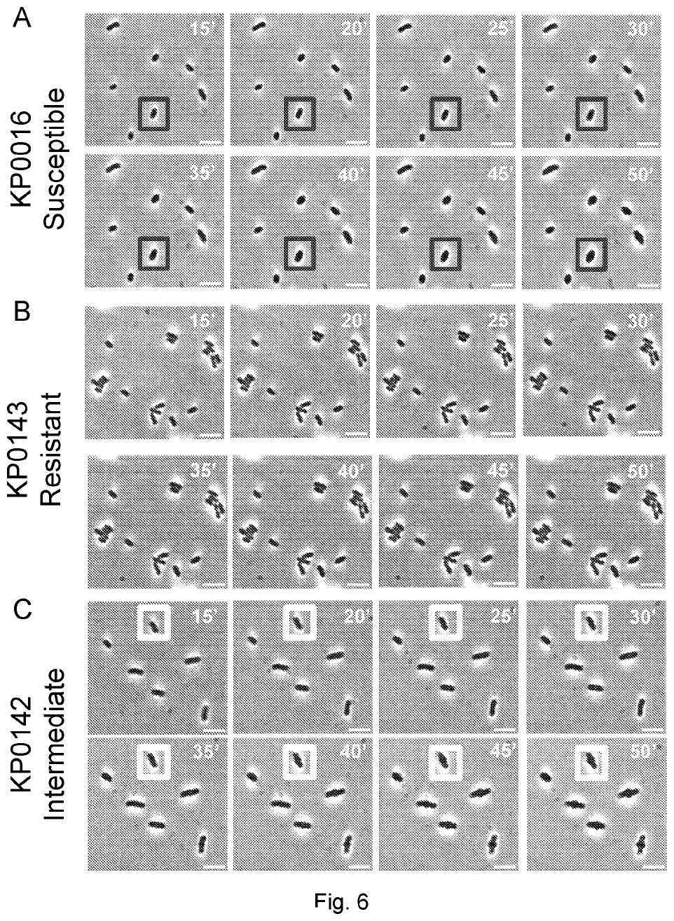

A total of 11 K. pneumoniae strains bearing various carbapenems resistance genes and sensitivity towards meropenem were monitored over time (Table 1). Changes in bacterial morphology were observed over 90 minutes post-antimicrobial incubation. Only the first 50 minutes, which is approximately the doubling time of K. pneumoniae, was analyzed. Each strain showed a unique morphological response to the varying antimicrobial concentration based on their MIC. FIG. 2A-B shows K. pneumoniae (KP0142) treated with and without meropenem. Bulging of the bacteria was only observed in the meropenem case. FIG. 6 compares three strains (KP0016-Susceptible, KP0143-Resistant, and KP0142-Intermediate) at the early time points. Similarly, at 5 μg/mL meropenem, which is higher than the breakpoint MIC for meropenem, susceptible and intermediate strains (KP0016 and KP0142) showed noticeable ‘bulging’ or protrusion around the center of the cell that was not observed in the resistant strain (KP0143).

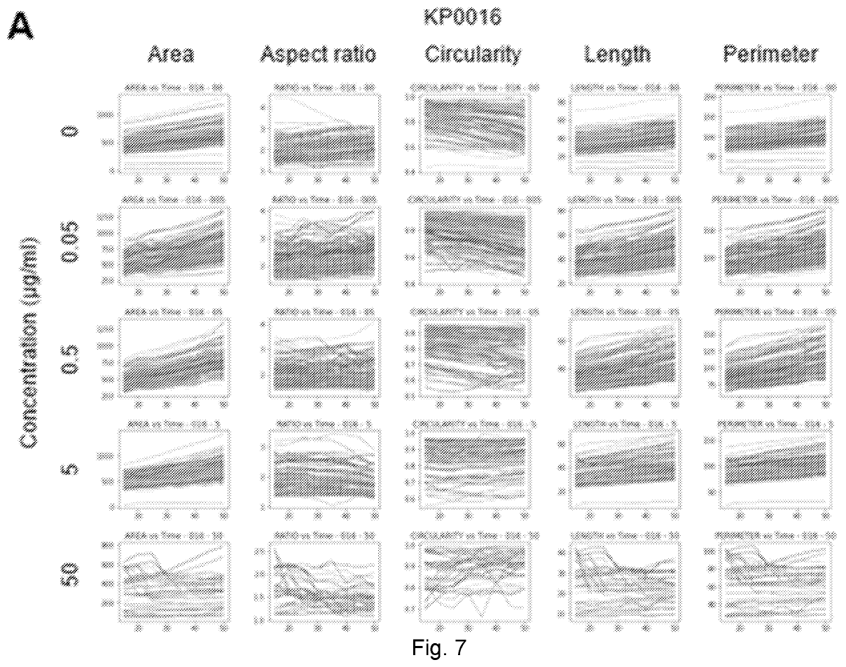

To automate the analysis and avoid subjectivity, the MicrobeJ plugin was applied for extracting morphological features (FIG. 2C). The cell behaviors were summarized by extracting the feature changing rates from the data (FIG. 2D and FIG. 7). FIG. 2E shows an example of the distributions of length and area changing rates of a single strain (KP0142) under various meropenem concentrations. The centroids of the feature changing rates changed with the antimicrobial concentration. However, there were large variations among individual bacteria at the same concentrations and substantial overlaps between different concentrations. It is challenging to accurately predict the bacteria response based on one or two features. A statistical approach, specifically machine learning algorithms, is required to analyze the multiparametric data for improving the classification accuracy.

Classification of Bacterial Response to Antimicrobial with Dynamic Features

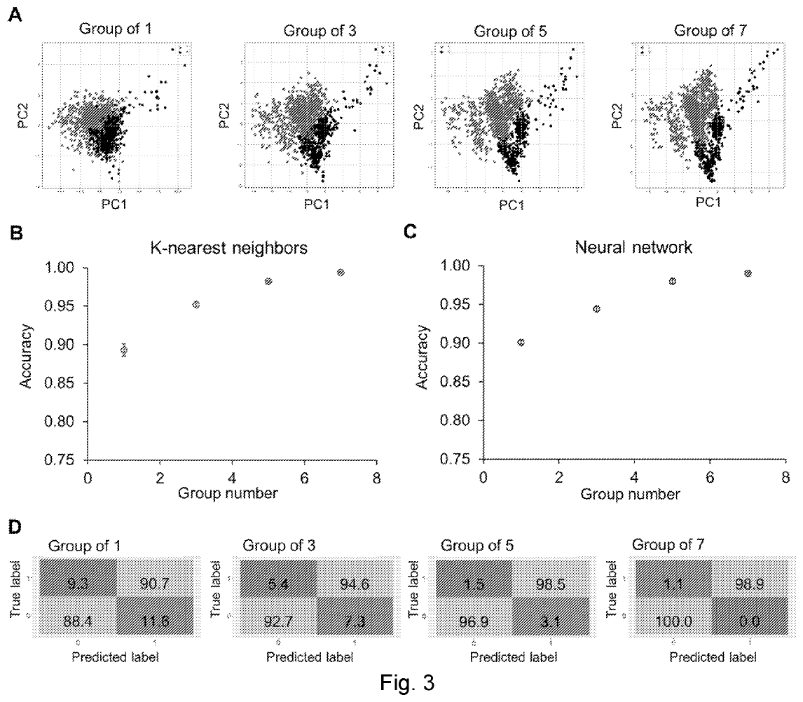

We first evaluated the dynamic (or time differential) approach for predicting the bacterial response to antimicrobial. The dynamic features of a total of 1338 bacterial cells across various concentrations were measured. The cells were labeled as 1 (resistant or division) or 0 (susceptible or no division) at the strain-concentration combination based on the CDC reported MIC of the bacteria. Since the behaviors of individual bacteria were highly diverse, one to seven bacteria under the same strain-concentration combinations were randomly grouped together. The average feature changing rates were calculated for each group. As shown in the principal component analysis (PCA) plots (FIG. 3A), grouping bacteria substantially reduced the variation within a group and increased the separation between the division and no division groups. The separation between the groups increased with the number of bacteria in the group. The grouped data were trained and validated using the training and validation datasets with the K-nearest neighbors and artificial neural network classifiers (FIG. 3B). For the 35-min data (i.e., 15-50 minutes of exposure), groups of one bacterium resulted in an accuracy of 89% and 90% with the K-nearest neighbors and artificial neural network classifiers, respectively. The prediction accuracy was generally improved by increasing the number of bacteria. With groups of seven bacteria, both classification algorithms reached over 99% accuracy. The improvement can be understood by a reduction of the statistical variation of individual cells by averaging data from multiple bacteria.

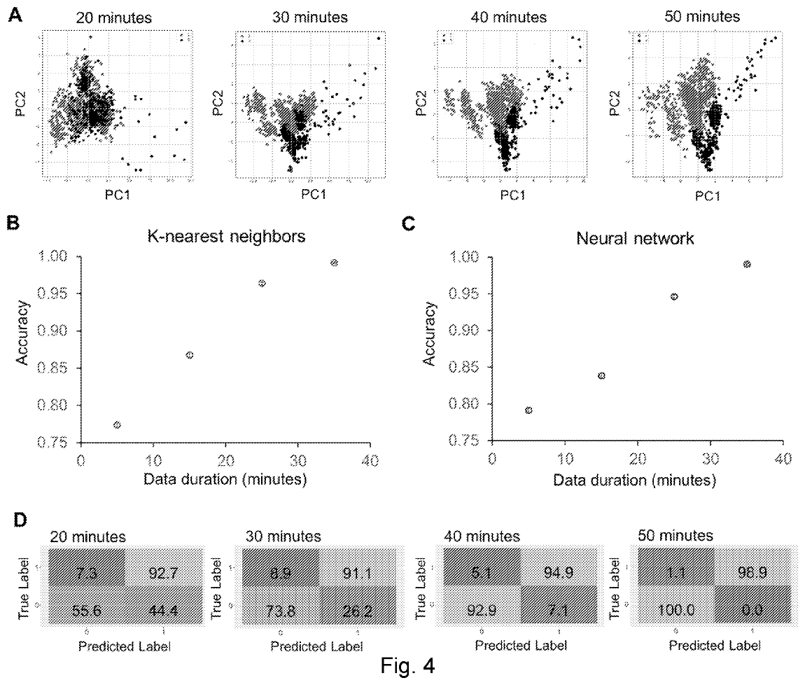

We then evaluated the accuracy of the classification model with sub-doubling times. The feature change rates were extracted using different durations of the data (5 to 35 minutes), corresponding to 15-50 minutes of antimicrobial incubation. Similarly, the data are summarized and visualized by the PCA plots (FIG. 4A). The data with a 5-min duration (i.e., between 15 minutes and 20 minutes after antimicrobial exposure) displayed a considerable variance and overlapped substantially. Nevertheless, the centroids of division and no division groups were distinct, and the separation improved with data of a longer duration. Clear separations between the groups could be observed in data with 25-min or 35-min durations. Again, we trained and validated the data using machine learning classifiers, including the K-nearest neighbors and artificial neural network algorithms (FIG. 4B-C). These algorithms exhibited similar performances and had an accuracy of ˜80% with the 5-min data. The results reached around 95% with the 25-min data and over 99% with the 35-min data. The confusion matrices indicated a false positive rate close to zero and a false negative rate of ˜1.1% (FIG. 4D). These results support the use of dynamic morphological features, i.e., the time differential approach, for predicting the antimicrobial response in a sub-doubling time.

Susceptibility Classification with Concentration Differential Features

### Discussion

This study demonstrated a rapid workflow for determining antimicrobial susceptibility of bacteria using time-lapse single cell imaging, computer vision, and machine learning models. By tracking the antimicrobial-induced morphological changes of individual cells, the MorphoAST workflow predicted the susceptibility category and MIC with a high accuracy in a fraction of the doubling time of the bacteria. The time differential strategy successfully predicted the susceptibility in as few as 20 minutes (two-fifths of the doubling time) of antimicrobial exposure with a high accuracy. The time differential approach with one or few concentrations could be useful when only a small number of bacteria is available (e.g., direct detection of bacteria from clinical samples) and measuring multiple antimicrobial concentrations is challenging (e.g., a point-of-care device that detects only a small number of conditions). When multiple concentrations are tested, the result can determine the minimum inhibitory concentration for comparing with susceptibility breakpoints. Our data also suggested that the prediction accuracy was generally improved with the antimicrobial exposure time, the number of bacteria being analyzed, and the number of testing conditions. These results underscore considerations and tradeoffs in the design of the single cell AST workflow and provide examples of future experimental designs.

Our results showed morphological features could enable useful information for rapid AST. In particular, the formation of bulges among K. pneumoniae with varying meropenem MIC are distinct and allowed us to classify susceptibility in a sub-doubling time. Bulge formation and cell lysis have been associated with the disruption of peptidoglycan and cell wall degradation prior to cell lysis. Prior studies have also reported the formation of cell-wall deficient spheroplasts in carbapenem-tolerant K. pneumoniae strains, after exposure to meropenem. While antimicrobial-induced shape changes in bacterial cells are not fully understood in general, recent quantitative modeling reveals potential advantages of this physiological adaptation, which include decreasing antimicrobial influx and diluting intracellular antimicrobials, leading to higher tolerance. The size and shape regulation differs from one organism to another but it provides a general reflection of their response in a short time frame. As demonstrated in this study, MorphoAST provides a workflow for utilizing the potential of morphological features for rapid AST. The workflow should be compatible with various morphological features, such as filamentation, bulging, and lysis, induced by specific antimicrobial-bacteria combination. The methodical procedure identifies and distinguishes morphological features that can be captured before and after the treatment (time differential) or with various antimicrobial dilutions (concentration differential).

A major advantage of the MorphoAST workflow is the short turnaround time. As the morphological features can be captured in a fraction of the doubling time, it dramatically reduces the assay time compared to the standard phenotypic AST (e.g., broth microdilution), which typically requires one or more days. The approach bypasses the requirement of cell replication in other single cell AST techniques. This characteristic will be particularly useful for diagnosing slow-growing and difficult-to-culture bacteria in normal laboratory conditions. Compared to other single cell analysis techniques that measure nanoscale motion of bacteria and metabolic activities, the MorphoAST workflow requires only a small inoculum size and a relatively simple setup consisting mainly agar pads and a microscope. As imaging is common in AST and low-cost microscopes are readily available, the MorphoAST workflow can be integrated with existing systems and implemented in a variety of settings. These advantages and characteristics will potentially increase the utility of MorphoAST for direct sample AST testing, especially in diseases like sepsis where the bacteria load in blood is typically very low. The small inoculum will also considerably reduce the time to AST results at the point of care.

MorphoAST will potentially accelerate microbiological analysis for combating multidrug-resistant bacteria, such as carbapenemase-producing Enterobacteriaceae. Further investigation will be required to elucidate the mechanisms of action of meropenem and its effects on other bacteria. Automation of the drug mixing and bacteria trapping steps will shorten the initial preparation time and capture changes in bacterial morphologies at earlier time points. Implementing the workflow in an integrated, low-cost imaging system, instead of a microscope, will also be useful for disseminating the workflow for managing a wide spectrum of infectious diseases.

### Minimum Inhibitory Concentration Prediction in Sub-Doubling Times

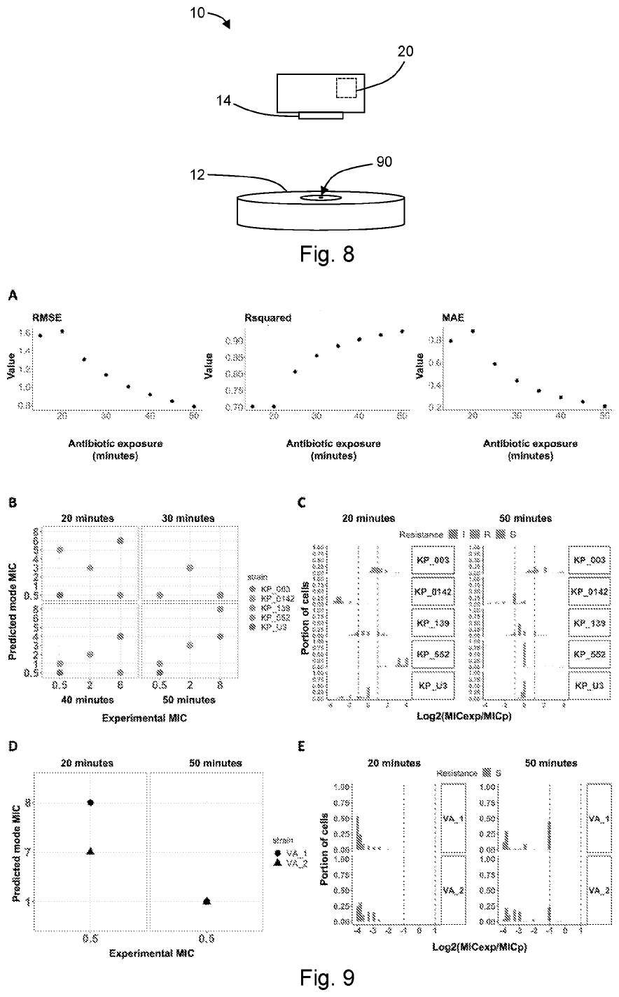

We further evaluated the use of the concentration differential data for MIC prediction. A Random Forest regressor was trained from a total of 39,135 cells across the concentrations. The model, including historic data (i.e., earlier time points), had a cumulative improvement in performance measured by the root-mean-square error (RMSE), R-squared, and mean absolute error (MAE) values with increased exposure time (FIG. 9A). The Random Forest regressor predicted the MIC in the 5-fold cross-validated training dataset with an RMSE of 0.8, MAE of 0.2, and an R² of 0.93. The performance of the model was assessed against 5 unseen KP strains, which comprised 6,067 cells. The experimental MIC and predicted MIC of the 5 unseen strains based on the Random Forest regressor are compared in FIG. 9B. The data showed a strong correlation, and the regressor collectively predicted the MIC within plus or minus, one two-fold dilution for all strains, resulting in an 80% essential agreement (EA) with 40 minutes of antimicrobial exposure that increases to 100% EA with 50 minutes of exposure. The predicted and experimentally reported MIC labels were also compared. Based on the predicted MIC for each strain, the model correctly predicted 100% (2/2) of the susceptible and intermediate (1/1) bacteria in as few as 30 minutes. The performance of the model increased with the antimicrobial exposure time and achieved 100% CA with 0% ME and 0% VME in 50 minutes. We then tested the model performance in predicting the MIC for individual cells within each population of the unseen strains tested (FIG. 9C). While there is heterogeneity in calls for individual cells from a single population, ˜79.9% of cells from 3/5 strains tested (KP 003, KP_139, and KP_U3) had predicted MICs within one-two fold dilution from the experimental MIC after 20 minutes of antimicrobial exposure. After 50 minutes of exposure, ˜85.1% of all cells from 5/5 tested strains had predicted MICs within one-two fold dilution from experimental MIC.

In addition to validation against unseen strains, the model was tested against imaging data from two clinical samples of KP obtained from patients visiting the VA (Palo Alto) with urinary tract infections (FIG. 9D-E). The experimental MICs for these clinical samples (VA_1 and VA_2) was 0.5 μg/mL, i.e., susceptible. Within 20 minutes of meropenem treatment, the model did not observe patterns for susceptibility it had learned from the training data and predicted both strains to be resistant with an MIC of 8 and 7 μg/mL respectively. When the incubation time was increased to 50 minutes, both strains were predicted to have a mode MIC of 1 μg/mL which accurately classifies both clinical isolates as susceptible with an MIC within one-two fold dilution from the experimental MIC resulting. Notably, heterogeneity within the population is more readily apparent with the clinical isolates where even after the 50 minutes of exposure, only a subset of the population started portraying susceptibility features as determined by the predicted MIC-˜45% of VA_1 and ˜23.3% of VA_2 had predicted MICs within one-two fold dilution with the mode MIC of 1 μg/mL. These results suggest a sufficient number of bacteria should be considered to compensate the heterogeneity of clinical samples in order to accurately predict the MIC.

Although the present disclosure has been described with respect to one or more particular embodiments, it will be understood that other embodiments of the present disclosure may be made without departing from the spirit and scope of the present disclosure.

What is claimed is:

1. A method for antibacterial susceptibility testing of a bacterium, comprising:
exposing a bacterium to an antimicrobial agent;
capturing a series of images of the bacterium over time after exposure, wherein the series of images are captured during an imaging period;
extracting, for each image of the series of images, values of each feature in a set of morphological features of the bacterium, wherein the set of morphological features comprises one or more of area, aspect ratio, length, circularity, perimeter, angularity, curvature, ferret, pole, roundness, sinuosity, width, trajectory, morphology, orientation, solidity, and z-score;
calculating a rate of change for each feature of the set of morphological features during the imaging period; and
determining an inhibition status of the bacterium using a machine-learning classifier applied to input data comprising the rate of change for each feature of the set of morphological features.

2. The method of claim 1, wherein the imaging period is less than the doubling time for the bacterium.

3. The method of claim 1, wherein the set of morphological features comprises area, aspect ratio, length, circularity, and perimeter.

4. The method of claim 1, wherein the machine-learning classifier is a k-nearest neighbor classifier.

5. The method of claim 1, wherein the machine-learning classifier is a multilayer perceptron classifier.

6. The method of claim 1, wherein the machine-learning classifier is a random forest regressor.

7. The method of claim 1, wherein the input data further comprises a concentration of the antimicrobial agent.

8. The method of claim 1, wherein the rate of change is calculated by fitting a curve of the values for a feature with an exponential function and calculating a coefficient.

9. The method of claim 8, wherein the exponential function is ƒ(t)=A1+A2e^{t/R}, where ƒ(t) is the feature as a function of time, A1 and A2 are constants, and R is the feature changing rate coefficient.

10. The method of claim 1, wherein the steps are repeated for various concentrations of antimicrobial agents.

11. A system for antibacterial susceptibility testing of a single-cell sample, comprising:
a sample holder;
an image sensor positioned to obtain one or more images of a sample held in the sample holder;
a processor in electronic communication with the image sensor, wherein the processor is configured to:
receive from the image sensor a series of images of a sample over time after exposure to an antimicrobial agent;
extract, for each image of the series of images, values of each feature in a set of morphological features of the sample, wherein the set of morphological features comprises one or more of area, aspect ratio, length, circularity, perimeter, angularity, curvature, ferret, pole, roundness, sinuosity, width, trajectory, and z-score;
calculate a rate of change for each feature of the set of morphological features during the imaging period; and
determine an inhibition status of the sample using a machine-learning classifier applied to input data comprising the rate of change for each feature of the set of morphological features.

12. The system of claim 11, wherein the imaging period is less than the doubling time for the bacterium.

13. The system of claim 11, wherein the set of morphological features comprises area, aspect ratio, length, circularity, and perimeter.

14. The system of claim 11, wherein the machine-learning classifier is a k-nearest neighbor classifier.

15. The system of claim 11, wherein the machine-learning classifier is a multilayer perceptron classifier.

16. The system of claim 11, wherein the machine-learning classifier is a random forest regressor.

17. The system of claim 11, wherein the input data further comprises a concentration of the antimicrobial agent.

18. The system of claim 11, wherein the processor is programmed to calculate the rate of change by fitting a curve of the values for a feature with an exponential function and calculating a coefficient.

19. The system of claim 18, wherein the exponential function is ƒ(t)=A1+A2e^{t/R}, where ƒ(t) is the feature as a function of time, A1 and A2 are constants, and R is the feature changing rate coefficient.

20. A non-transitory computer-readable medium having stored thereon a computer program for instructing a computer, the computer program comprising:
receive from an image sensor a series of images of a sample over time after exposure to an antimicrobial agent;
extract, for each image of the series of images, values of each feature in a set of morphological features of the sample, wherein the set of morphological features comprises one or more of area, aspect ratio, length, circularity, perimeter, angularity, curvature, ferret, pole, roundness, sinuosity, width, trajectory, and z-score;
calculate a rate of change for each feature of the set of morphological features during the imaging period; and
determine an inhibition status of the sample using a machine-learning classifier applied to input data comprising the rate of change for each feature of the set of morphological features.
